(19) United States
(12) Patent Application Publication
(10) Pub. No.: US 2015/0297073 A1
(43) Pub. Date: Oct. 22, 2015

(54) ARTHROSCOPIC INSTRUMENT ASSEMBLY, AND METHOD OF LOCALIZING MUSCULOSKELETAL STRUCTURES DURING ARTHROSCOPIC SURGERY

(71) Applicant: ACADEMISCH MEDISCH CENTRUM, Amsterdam (NL)

(72) Inventors: Duy Tan Nguyen, Amsterdam (NL); Ton G Van Leeuwen, Amsterdam (NL); Pepijn Van Horssen, Amsterdam (NL)

(21) Appl. No.: 14/443,828

(22) PCT Filed: Nov. 18, 2013
(86) PCT No.: PCT/EP2013/074115

§ 371 Date: May 19, 2015

Related U.S. Application Data
(60) Provisional application No. 61/727,976, filed on Nov. 19, 2012.

(30) Foreign Application Priority Data
Nov. 19, 2012 (EP) 12193157.0

(51) Int. Cl. A61B 1/317; A61B 1/05; A61B 1/06; A61B 1/07; A61B 1/00; A61B 1/04

(52) U.S. Cl. CPC A61B 1/317; A61B 1/00045; A61B 1/043; A61B 1/0638; A61B 1/0676; A61B 1/00078; A61B 1/00009; A61B 1/00011; A61B 1/07; A61B 1/0684; A61B 1/05

(57) ABSTRACT

An arthroscopic instrument assembly (100), comprising: an illumination system (120) for illuminating an operative field, including a light source (122a) configured to produce light having at least one ligament excitation wavelength; an arthroscope (110); an image transmission system (130) configured to transmit a fluorescent image of the operative field at a distal end (112b) of the arthroscope (110) to an image viewing system (150); an image processing system (140) configured to process the fluorescent image as it passes through the image transmission system, so as to provide a false-color fluorescent image of the operative field in which a contrast between ligament and bone structures present in the operative field is enhanced relative to the unprocessed fluorescent image; and an image viewing system (150), operably connected to the image transmission system (130), and including a display (152) configured to enable viewing of the false-color fluorescent image.

### FIELD OF THE INVENTION

The present invention relates to an arthroscopic instrument assembly, and to a method of localizing musculoskeletal tissue structures in a joint during arthroscopic surgery.

### BACKGROUND

The anterior cruciate ligament (ACL) is the most commonly injured ligament of the knee. In cases where the injury involves a complete disruption of the ACL arthroscopic surgery may be required to reconstruct the ACL.

During a surgical reconstruction, a torn ACL may be replaced by a graft, such as a tendon transplant, that is inserted into the knee. To fully restore the prior knee function without pain, instability and/or development of degenerative changes, it is of paramount importance that the graft is properly affixed to the tibia (shin bone) and the femur (thigh bone), in particular within the respective native attachment sites of the ACL. However, despite the fact that the anatomical position of the ACL has been geometrically described and charted relative to arthroscopically visible landmarks of the tibia and the femur in various studies, accurate attachment of the graft remains difficult. This may be at least partially due to the limited, two-dimensional view provided by an arthroscope, which appears to render the aforementioned descriptions and landmarks insufficient for correctly positioning the graft during arthroscopic surgery.

### SUMMARY OF THE INVENTION

It is an object of the present invention to provide for an arthroscopic instrument assembly that facilitates the localization of the native attachment sites of the ACL within a knee joint during arthroscopic surgery.

It is another object of the present invention to provide for a method of localizing ligament structures, such as native attachment sites of the ACL, within a joint, such as a knee joint, during arthroscopic surgery.

To this end, a first aspect of the present invention is directed to an arthroscopic instrument assembly for viewing an operative field inside a joint.

The assembly may comprise an illumination system for illuminating the operative field, including a light source configured to produce light having at least one ligament excitation wavelength. The assembly may also comprise an arthroscope defining a rigid tubular housing extending between a proximal operator end and a distal operative field end, and an image transmission system, that is at least partly accommodated by the tubular housing, and configured to transmit a fluorescent image of the operative field at the distal end of the tubular housing to an image viewing system. The assembly may further comprise an image processing system, incorporated in the image transmission system, and configured to process the fluorescent image of the operative field as it passes through the image transmission system, so as to provide a false-color fluorescent image of the operative field in which a contrast between ligament and bone structures present in the operative field is enhanced relative to the unprocessed fluorescent image, in a limiting case possibly such that only one of the ligament and bone structures, preferably the ligament structures, is still visible. An image viewing system may be operably connected to the image transmission system, and include a display configured to enable viewing of the false-color fluorescent image of the operative field.

A second aspect of the present invention is directed to a method for discriminating between at least ligament and bone tissues, and hence for facilitating the localization ligament structures within an operative field inside a joint. The method may include illuminating the operative field with light having at least one ligament excitation wavelength, and acquiring and transmitting a fluorescent image of the operative field to an image viewing system. The method may also include processing the acquired fluorescent image of the operative field as it is transmitted to the image viewing system, so as to generate a false-color fluorescent image of the operative field in which a contrast between ligament and bone structures present in the operative field is enhanced relative to the unprocessed fluorescent image, in a limiting case possibly such that only one of the ligament and bone structures, preferably the ligament structures, is still visible. The method may further include viewing the false-color fluorescent image on the image viewing system, and localizing the ligament structures present within the operative field in the false-color fluorescent image.

These and other features and advantages of the invention will be more fully understood from the following detailed description of certain embodiments of the invention, taken together with the accompanying drawings, which are meant to illustrate and not to limit the invention.

### BRIEF DESCRIPTION OF THE DRAWINGS

FIG. 1 schematically illustrates the anatomy of a human knee;

FIG. 2 is a graph schematically illustrating the emission spectra of ACL tissue and bone tissue in a bovine knee joint for an excitation wavelength of 280 nm; and

FIG. 3 schematically illustrates an exemplary embodiment of the arthroscopic instrument assembly according to the present invention.

FIG. 4 is a graph showing the normalized difference between the emission spectrum of bone divided by emission spectrum of the ACL as a function of the excitation wavelength.

### DETAILED DESCRIPTION

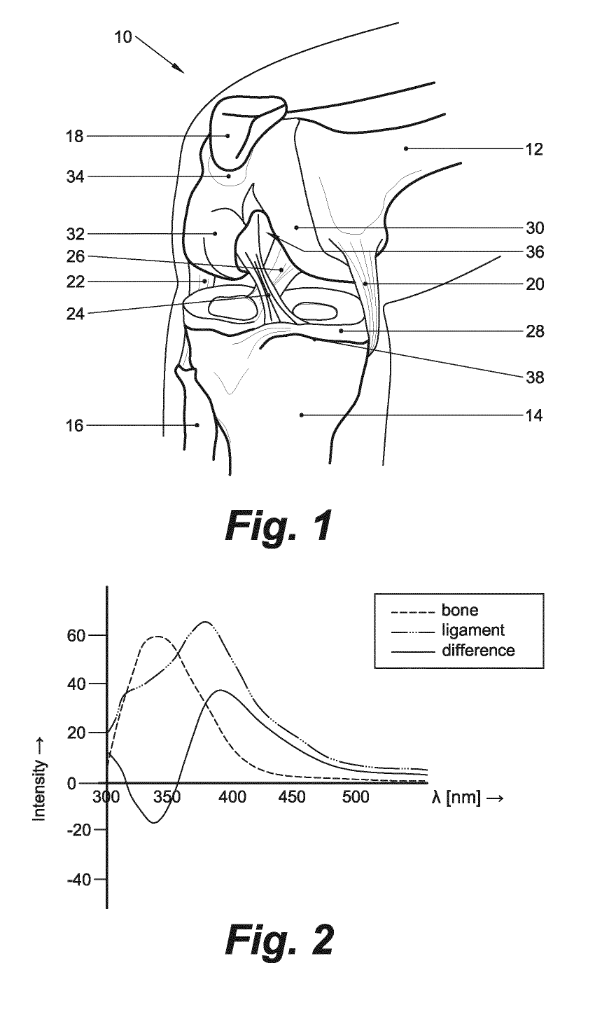

FIG. 1 schematically illustrates a human knee 10 in an approximately 90° flexed condition. The knee 10 is made up of four major bones: the femur (thigh bone) 12, the tibia (shin bone) 14, the fibula (outer shin bone) 16, and the patella (knee cap) 18. The fibula 16 is a relatively thin bone that lies at the outside of the tibia 14 and travels right down to the ankle joint. The other three bones define two articulations, one between the femur 12 and the tibia 14, and one between the femur 12 and the patella 18. To this end, the lower end of the femur 12 defines two condyles (i.e. rounded prominences), the medial (inner) femoral condyle 30 and the lateral (outer) femoral condyle 32, which articulate with the tibial plateau, i.e. the generally flat upper portion of the tibia 14. Anteriorly, the femoral condyles 30, 32 are slightly prominent and separated from one another by a smooth, shallow articular depression called the patellar surface 34, which articulates with the patella 18. Posteriorly, the condyles 30, 32 project considerably, and the interval between them forms a deep notch called the intercondylar fossa 36.

In addition to the bones 12, 14, 16, 18, the anatomy of the knee 10 includes a meniscus 28, and a number of ligaments including the medial collateral ligament (MCL) 20, the lateral collateral ligament (LCL) 22, the anterior cruciate ligament (ACL) 24 and the posterior cruciate ligament (PCL) 26. The meniscus 28, which defines two crescent-shaped menisci lying respectively on the medial and lateral edges of the tibial plateau 38, acts as a shock absorber for the knee 10. The ligaments 20, 22, 24, 26 provide the knee 10 with much of its stability, wherein each serves to provide stability in one or more of a variety of different knee positions. The cruciate ligaments 24, 26 cross each other in the middle of the knee joint 10. The PCL 26 travels from the posterior of the tibia 14 to the anterior of the femur 12, while the ACL 24 travels from the anterior of the tibia 14 to the posterior of the femur 12. More specifically, the ACL 24 attaches to the femur 12 on a posteromedial surface of the lateral femoral condyle 32 within the intercondylar fossa 36. On the tibia 14, the ACL 24 attaches anterolateral to the anterior tibial spine, a bony ridge in the middle of the tibial plateau 38.

The ACL 24 is the most commonly injured ligament of the knee 10. An ACL injury, such as an over-stretched or disrupted ACL, may, for instance, be sustained by twisting of the knee 10, and cause serious instability thereof. Whereas minor tears in the ACL 24 may heal over time, larger tears in the ACL and a completely disrupted ACL require arthroscopic surgery.

During a surgical reconstruction of a completely disrupted ACL 24, a graft, e.g. a tendon transplant, may be inserted into the knee 10 to replace the ACL. To regain prior function without pain, instability and/or the development of degenerative changes, it is of paramount importance that the graft is properly affixed to the tibia 14 and the femur 12, in particular within the respective native attachment sites of the ACL 24. However, despite the fact that the anatomical position of the ACL 24 has been geometrically described and charted relative to arthroscopically visible landmarks, accurate attachment of the graft remains difficult. This may at least in part be due to the limited, two-dimensional view provided by an arthroscope, which may render the landmarks and descriptions provided by the aforementioned studies insufficient for correctly positioning ACL during arthroscopic surgery.

Presently disclosed is an arthroscopic instrument assembly that greatly facilitates the localization of the native attachment sites of the ACL 24 during a surgical procedure, in particular by providing its operator with false-color fluorescent images of the operative field inside the knee in which the visibility of the native attachment sites is enhanced. The composition of the false-color images may be based on differences in the fluorescent properties between the ACL 24, and specifically the ACL's end portions by means of which it attaches to the tibia 14 and the femur 12, on the one hand, and bone tissue on the other. To this end, the invention envisages two primary imaging methods, each of which may be independently implemented in the arthroscopic instrument assembly. Below, these imaging methods are briefly discussed in turn.

The first imaging method makes use of the experimental finding that the emission spectra of ACL tissue and bone tissue exhibit marked differences for excitation wavelengths in the range of 260-300 nm. By way of illustration, FIG. 2 schematically shows the emission spectra of ACL tissue and bone tissue in a bovine knee joint for an excitation wavelength of 280 nm. The intensity curve reflecting the emission spectrum of the ACL tissue is labelled “ligament”, while the intensity curve reflecting the emission spectrum of the bone tissue is labelled “bone”. A curve reflecting the difference between the two intensity curves “ligament” and “bone” is also shown, and labelled “difference”. As can be seen in the graph of FIG. 2, a local minimum of the difference curve may be found in the emission wavelength range 325-345 nm, while a local maximum of the difference curve may be found in the emission wavelength range 370-450 nm. Although the actual maximum difference may be found closely around an emission wavelength of 390 nm, the emission wavelength range of 400-450 nm is of particular interest as the emission intensity of bone tissue in this range rapidly declines with increasing emission wavelength. Accordingly, by illuminating the approximate locations of a torn ACL's native attachment sites within a knee joint with light having an excitation wavelength in the range of 260-300 nm, and preferably in the range of 270-280 nm, and imaging the illuminated locations through fluorescent light at emission wavelengths in the range of 400-450 nm, the ACL's native attachment sites may be made visible substantially exclusively. Where desired, an intensity threshold may be applied to filter or block out fluorescent emission contributions from the bone tissue.

The second imaging method makes use of the experimental finding that the ligament and bone tissues making up a knee joint have mutually different emission spectra for various excitation wavelengths. Although the various emission spectra in themselves may not enable the ACL's native attachment sites to be exclusively imaged at a certain emission wavelength, as in the first imaging method, the differences in the various spectra may be used to distinguish between the types of tissue by means of a spectral unmixing procedure.

The spectral unmixing procedure, which in itself may be known in the art, may rely on at least two fluorescent images taken at different emission wavelengths for which an intensity ratio between the intensity of ligament tissue and the intensity of bone tissue is different. In one embodiment, spectral unmixing may be performed by storing the relative intensities of each tissue type in a matrix, and multiplying the inverse of the matrix with the acquired fluorescent images to obtain the isolated contributions of the respective tissue types. This may be understood as follows.

If I_{α} is a fluorescent image taken at emission wavelength α, and I_{β} is a fluorescent image taken at emission wavelength β, both images I_{α}, I_{β} may be described as the superposition of an individual color-component contribution matrix matrix C_{ACL} (relating to the ligament tissue in isolation) and an individual color component contribution matrix C_{Bone} (relating to the bone tissue in isolation), each times a respective intensity factor a-d

I_{α}=a·C_{ACL}+b·C_{Bone}

I_{β}=c·C_{ACL}+d·C_{Bone}. Eq. (1)

Eq. (1) may be recast in matrix notation as:

$$\begin{matrix}I = A C & Eq . ( 2 ) \\ wherein & \\ I = ( \begin{matrix}{I}_{α} \\ {I}_{β}\end{matrix} ) , A = [ \begin{matrix}a & b \\ c & d\end{matrix} ] , and C [ \begin{matrix}{C}_{ACL} \\ {C}_{Bone}\end{matrix} ] . & Eq . ( 3 )\end{matrix}$$

Eq. (2) may be rewritten to express that the individual color component contribution matrix C is obtainable by multiplying the inverse A⁻¹ of the intensity factor matrix A by the composite fluorescent image matrix I:

C=A⁻¹I Eq. (4)

For the 2×2 matrix A, the inverse A⁻¹ is straightforward and may be written as:

$${A}^{- 1} = \frac{1}{( ad - bc )} [ \begin{matrix}d & - b \\ - c & a\end{matrix} ] \qquad Eq . ( 5 )$$

By combining Eqs. (3), (4) and (5), the following expressions for the individual color component contribution matrices of the ligament issue and bone tissue C_{ACL}, C_{Bone} may be obtained:

$$\begin{matrix}{C}_{ACL} = \frac{1}{( ( ad - bc ) ( d \cdot {I}_{α} - b \cdot {I}_{β} ) )} & Eq . ( 6 ) \\ {C}_{Bone} = \frac{1}{( ( ad - bc ) ( a \cdot {I}_{β} - c \cdot {I}_{α} ) )} & Eq . ( 7 )\end{matrix}$$

Eq. (6) and Eq. (7) are subject to the condition that ad≠bc, or a/b≠c/d, meaning that the intensity ratios between ligament tissue and bone tissue are different for the emission images I_{α} and I_{β}.

Optimal spectral unmixing is achievable when both (i) a difference in intensity between the tissue types (e.g. |a−b|, and |c−d|) in each of the at least two fluorescent images I_{α}, I_{β} is large, preferably such that the intensities of the tissue types are generally opposite in the two fluorescent images, i.e. such that the intensity of ligament tissue is greater than that of bone tissue in one image, while the intensity of ligament tissue is smaller than that of bone tissue in the other image, and (ii) a difference in tissue type intensity ratios between the at least two fluorescent images (e.g. |(a/b)−(c/d)|) is large.

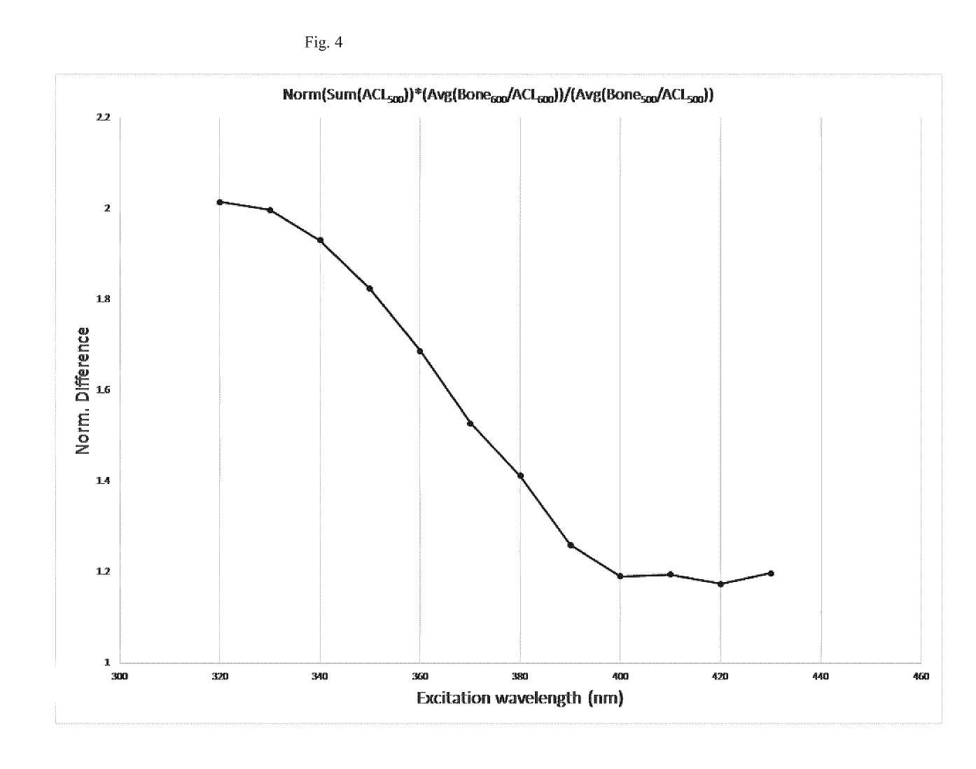

Although fluorescent images with sufficiently large differences in tissue type intensities and tissue type intensity ratios may be obtained for most if not all excitation wavelengths within the near and middle ultraviolet ranges (i.e. 200-400 nm), optimal conditions for spectral unmixing have been identified for only two excitation wavelength subranges: 300-350 nm and 380-395 nm. More preferably, said excitation wavelength is lower than 395, 394, 393, 392, 391 nm, as below this wavelength better identification of the ACL may be achieved. This is illustrated in FIG. 4. For the excitation wavelength subrange of 300-350 nm, correspondingly suitable emission wavelengths have been found at 390±20 nm and 460±20 nm. For the excitation wavelength subrange of 380-395 nm, more preferably 380-394 nm, corresponding large difference in tissue type intensities and tissue type intensity rations have been found at emission wavelengths of 500±20 nm, showing primarily ligament tissue, and of 600±20 nm, showing primarily bone tissue. Use of the emission wavelength subrange of 380-395 nm may be preferred over that of 300-350 nm, as it may be easier and more economical to implement, in particular because it is safer from a human perspective and requires less complex optics. It is noted that it has also proven possible to satisfactorily enhance the visibility of ligament tissue by means of a spectral unmixing procedure based on the red, green and blue components of a fluorescent image captured with a standard RGB-camera.

The below table summarizes the characteristics of the two primary imaging methods:

TABLE 1
Summary of primary imaging methods

| | Excitation light | Emission imaging | |
|---|---|---|---|
| First | 260-300 nm, | filtering of fluorescent light | direct exclusive |
| method | preferably: | filter: 400-450 nm | imaging of |
| | 270-280 nm | | ligament tissue |
| Second | near/middle-UV | filtering + spectral unmixing | postprocessing |
| method | (i.e. 200-400 nm, | of fluorescent light | by spectral |
| | 200-394 nm), | RGB-filtering possible; | unmixing |
| | preferably: | preferred filters: | to achieve |
| | 300-350 nm, or | 390 ± 20 nm/460 ± 20 nm, | exclusive |
| | 380-395 nm | resp. 500 ± 20 nm/ | imaging |
| | | 600 ± 20 nm | of ligament |
| | | | tissue |

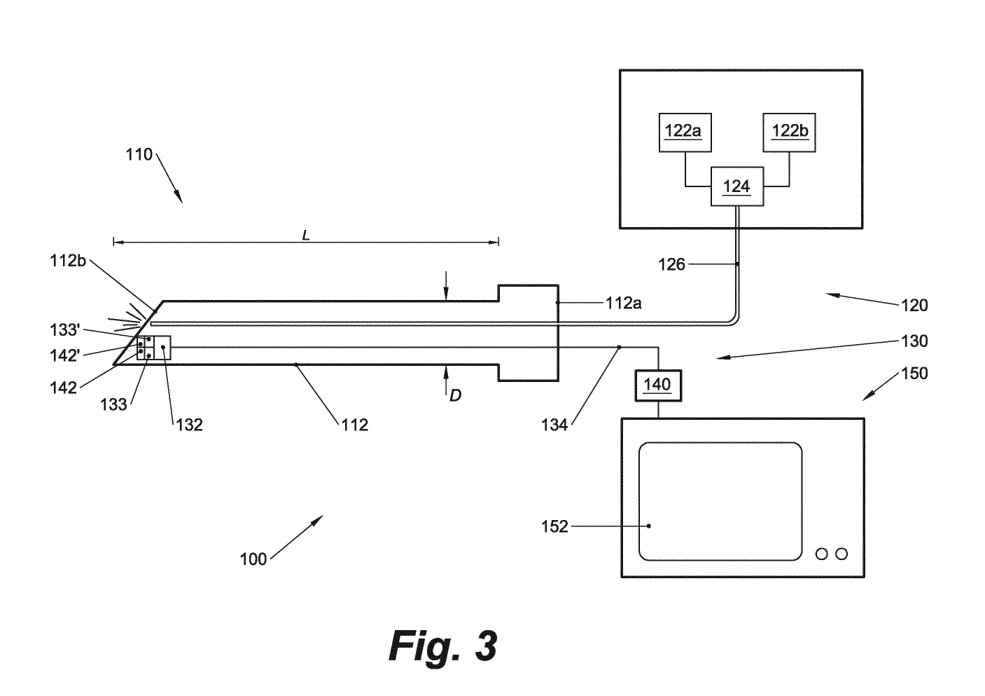

Now that the underlying methodology for imaging and localization of the ACL's native attachment sites has been clarified, attention is invited to the construction of the arthroscopic instrument assembly according to the present invention. FIG. 3 schematically illustrates an exemplary embodiment of such an assembly 100.

The arthroscopic instrument assembly 100 may include an arthroscope 110. The arthroscope 110 may define a rigid tubular housing or cannula 112, which may extend between a proximal operator end 112a and a distal operative field end 112b. The latter end 112 may be slanted, i.e. cut at an angle, as shown. The rigid tubular housing 112 may typically have a length L equal to or less than 18 cm, an outer diameter D equal to or less than 5 mm, and accommodate portions of an illumination system 120, an image transmission system 130 and/or an image processing system 140, as will be clarified below.

In some embodiments, the arthroscopic instrument assembly 100 may include a resilient, tubular introducer sheath (not shown) within which the arthroscope 110 may be sheathed during a surgical procedure in order to protect a patient from injury. The introducer sheath may have a length slightly greater than that of the rigid tubular housing 112 of the arthroscope 110, and an outer diameter that is up to about 2 mm greater than that of the rigid tubular housing 112 of the arthroscope 110. During use, irrigation fluid may be supplied to and/or discharged from the operative field through an irrigation channel that is at least partly defined by the introducer sheath and/or the housing 112 of the arthroscope, so as to irrigate the operative field and maintain a clear view.

The arthroscopic instrument assembly 100 may further comprise an illumination system 120 for illuminating the operative field. In a preferred embodiment, the illumination system 120 may enable illumination of the operative field in at least two simultaneously or alternatively selectable illumination modes. In a first illumination mode, the illumination system may enable illumination of the operative field with light capable of fluorescently exciting the tissue that makes up the knee joint, so as to allow for the generation of typically false-color fluorescent images thereof in which the visibility of in particular ligament tissue may be enhanced. In a second illumination mode, the illumination system 120 may enable illumination of the operative field with generally white light that allows for the capture or generation of typically real-color images of the operative field, and thereby for plain visual inspection of the tissues present therein.

In an implementation of the illumination system 120 capable of the first illumination mode, the illumination system 120 may include a first light source 122a configured to produce light having a wavelength in a ligament excitation wavelength range, and thus capable of fluorescently exciting ligament tissue in the human or animal body. Light with this capability may generally be found in a wavelength range of 200-520 nm. In preferred embodiments, however, excitation of ligament tissue may be effected using invisible light in the near and middle ultraviolet ranges of wavelengths, i.e. the range of 200-400 nm, so as to prevent overlap between the excitation spectrum and usable portions of the visible emission spectrum. More specifically, in embodiments of the arthroscopic instrument assembly 100 based on the first imaging method discussed above, the first light source 122a may be configured to produce light having a wavelength in the range of 260-300 nm, and preferably in the range of 270-280 nm, while embodiments based on the second imaging method may include a first light source 122a configured to produce light having a wavelength in the range of 300-400 nm, and preferably in the range of 380-395 nm, more preferably 394 nm or lower.

The first light source 122a may in principle have any suitable construction. In one embodiment, for example, the first light source 122a may comprise a (high-power) LED. In another embodiment, the first light source 122a may comprise a gas discharge lamp. Both the LED and the gas discharge lamp may optionally be used in combination with a suitable optical bandpass filter. An embodiment configured to implement the second imaging method, for instance, may thus include a xenonlamp with a 395 nm (10 nm-bandwidth) filter.

In an implementation of the illumination system capable of the second illumination mode, the illumination system may include a second light source 122b configured to produce generally white light, i.e. light having a spectrum that substantially covers the wavelength range 400-700 nm, or at least includes blue, green and red colors. Like the first light source 122a, the second light source 122b may in principle have any suitable construction, and for example include one or more LEDs.

In one embodiment of the arthroscopic instrument assembly 100, the illumination system 120 may be accommodated in a separate, stand-alone light probe that is not structurally connected to the arthroscope 110. In a preferred embodiment, however, the illumination system 120 may be at least partially integrated into the arthroscope 110. In one such preferred embodiment, such as that illustrated in FIG. 3, the first and/or second light sources 122a, 122b may themselves be disposed outside of the arthroscope 110, while a light guide 126, e.g. a quartz fiber optic light guide, may be connected to the first and/or second light sources 122a, 122b, and extend therefrom through the tubular housing 112 of the arthroscope 110, into the distal end 112b thereof. The first and second light sources 122a, 122b may be associated with their own, dedicated light guide, or share a common light guide 126, which may be connected to the light sources 122a, 122b through a operator-controllable optical switch 124 that enables the operator to alternatively couple light from the first and second light sources 122a, 122b into the light guide 126. In another such preferred embodiment, the first and/or second light sources 122a, 122b may themselves be wholly or partly accommodated in the arthroscope 110. This may be particularly practical in case the first and/or second light sources 122a, 122b are implemented in the form of one or more relatively small LEDs, which may be incorporated into the distal end or tip 112b of the arthroscope 110.

The arthroscopic instrument assembly 100 may further comprise an image transmission system 130 configured to transmit a fluorescent image of the operative field at the distal end 112b of the tubular housing 112 to an image viewing system 150. The image transmission system 130 may typically include a digital camera 132 having at least one image sensor 133, 133′ that records or captures optical images in electronic form. The camera/at least one image sensor 133, 133′ may be accommodated in the distal end 112b of the tubular housing 112 of the arthroscope 110, or be disposed external to the arthroscope 110. In the former case, which is illustrated in FIG. 3, an electric camera signal cable 134 of the image transmission system 130 or a suitable alternative connection, e.g. a wireless connection, may operably connect the camera 132 to the image viewing system 150; in the latter case, the camera 132 may additionally include an optical guide (not shown) that extends between the camera/at least one image sensor 133, 133′ and the distal end 112b of the tubular housing 112 of the arthroscope 110, so as to transmit the image from said distal end 112b to the at least one image sensor.

The image transmission system 130 may incorporate an image processing system 140 that is configured to process the fluorescent image of the operative field as it passes through the image transmission system 130, in order to provide a false-color fluorescent image of the operative field in which a contrast between ligament and bone structures present in the operative field is enhanced relative to the unprocessed fluorescent image.

In one embodiment, the image processing system 140 may include at least one optical bandpass filter 142. The optical bandpass filter 142 may be of any suitable type, and be based on any suitable physical principle. The optical bandpass filter 142 may, for instance, include an absorbing glass filter, dye filter, or color filter that is based on the wave-length dependent absorption in some material such as a glass dopant, dye, pigment or semiconductor. Alternatively or in addition, the optical bandpass filter may include a tunable optical bandpass filter, such as a liquid crystal tunable filter (LCTF), in which a liquid crystal may be electronically controlled to select wavelengths of light to be transmitted. The optical bandpass filter 142 may be incorporated into the camera 132 of the image transmission system 130, and be positioned upstream or in front of of its image sensor, such that it is disposed in an optical path of the image transmission system. In an embodiment implementing the first imaging method, the camera 132 may typically include one image sensor and one optical bandpass filter 142 that is associated therewith. In an embodiment implementing the second imaging method, the camera 132 may include one or two image sensors: e.g. one (RGB-) image sensor in case spectral unmixing is to be performed on the red, blue and green components of the camera signal, and two (or more) image sensors in case spectral unmixing is to be performed on two fluorescent images acquired simultaneously but at different emission wavelengths. In the former case, the single image sensor need not be associated with a separate optical bandpass filter 142 as the sensor itself may act as three filters; it may be desired, however, to use a long-pass optical bandpass filter with a lower cut-off wavelength in the range of about 430±10 nm in order to minimize the over exposure of the blue channel of the image sensor by the first light source 122a. In the latter case, each of the image sensors of the camera 132 may be associated with a respective optical bandpass filter 142.

On the basis of the foregoing discussion of imaging methods it will be clear that the image processing system 140 in an embodiment implementing the first imaging method may include an optical bandpass filter for wavelengths in the range of 400-450 nm, for instance a 410 nm (10 nm-bandwidth) optical bandpass filter, while the image processing system in an embodiment implementing the second imaging method by means of two image sensors may include two optical bandpass filters, e.g. a 500 nm (20 nm bandwidth) and a 600 nm (20 nm bandwidth) optical bandpass filter in case an excitation wavelength in the range of 380-395 nm is used, or, alternatively, a 390 nm (20 nm bandwidth) and a 460 nm (20 nm bandwidth) optical bandpass filter in case an excitation wavelength in the range of 300-350 nm is used.

In a preferred embodiment, in particular an embodiment configured to provide both the first and the second illumination mode, the at least one optical bandpass filter 142 may not be permanently active in the optical path of the image transmission system 130. Instead, the image processing system 140 may include an optical bandpass filter activation/deactivation device (not shown) that effectively enables activation and deactivation of the at least one optical bandpass filter 142, such that, in a deactivated condition of the filter(s), white light may enter and/or pass through the image transmission system 130 unfiltered.

In embodiments based on the second imaging method, the image processing system 140 may further include a processor configured to perform the spectral unmixing of the various fluorescent images of the operative field. The processor may typically be disposed downstream of the image sensors of the digital camera 132, and thus act upon the electric signals outputted by the image sensors.

As mentioned, the arthroscopic instrument assembly 100 may also include an image viewing system 150 that is operably connected to the image transmission system 130, and configured to enable viewing of the false-color fluorescent image of the operative field. Structurally, the image viewing system 150 may include a display or monitor 152. The display 152 may preferably be a high-definition color display, although for instance a black and white display may also be usable.

The construction and operation of the presently disclosed arthroscopic instrument assembly and method have been described above with reference to a knee joint, and in particular for the purpose of performing reconstructive surgery on the ACL. It is understood, however, that although the arthroscopic instrument assembly and method are particularly suited for this application, they are not limited thereto. Both the arthroscopic instrument assembly and the method of localizing ligament tissue within an operative field may be used in other joints than the knee, human or animal.

With regard to the terminology used in this text, the following is noted. The term ‘false-color fluorescent image’ may be construed to refer to a fluorescent image that depicts its subject, in particular a portion of the operative field, in colors that differ from those a full-color fluorescent image, indiscriminately containing emission wavelengths/colors across the entire visible spectrum, would show. Accordingly, a fluorescent image generated through processing, e.g. filtering certain wavelengths therefrom and/or color to grayscale conversion, is understood to be a ‘false-color fluorescent image’.

Although illustrative embodiments of the present invention have been described above, in part with reference to the accompanying drawings, it is to be understood that the invention is not limited to these embodiments. Variations to the disclosed embodiments can be understood and effected by those skilled in the art in practicing the claimed invention, from a study of the drawings, the disclosure, and the appended claims. Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, it is noted that particular features, structures, or characteristics of one or more embodiments may be combined in any suitable manner to form new, not explicitly described embodiments.

### LIST OF ELEMENTS

10 human knee
12 femur (thigh bone)
14 tibia (shin bone)
16 fibula (outer shin bone)
18 patella (knee cap)
20 medial collateral ligament (MCL)
22 lateral collateral ligament (LCL)
24 anterior cruciate ligament (ACL)
26 posterior cruciate ligament (PCL)
28 meniscus
30 medial femoral condyle
32 lateral femoral condyle
34 patellar surface
36 intercondylar fossa
38 tibial plateau
100 arthroscopic instrument assembly
110 arthroscope
112 rigid tubular housing
112a, b proximal operator end (a) and distal operative field end (b) of tubular housing
120 illumination system
122a, b first (a) and second (b) light source
124 optical switch
126 light guide
130 image transmission system
132 digital camera
133, 133′ image sensor
134 electric signal cable
140 image processing system
142, 142′ optical bandpass filter
150 image viewing system
152 display
L length of rigid tubular housing of arthroscope
D outer diameter of rigid tubular housing of arthroscope

1. An arthroscopic instrument assembly for viewing an operative field inside a joint, comprising:
an illumination system for illuminating the operative field, including a light source configured to produce light having at least one ligament excitation wavelength;
an arthroscope defining a rigid tubular housing extending between a proximal operator end and a distal operative field end;
an image transmission system, at least partly accommodated by the tubular housing, and configured to transmit a fluorescent image of the operative field at the distal end of the tubular housing to an image viewing system;
an image processing system, incorporated m the image transmission system, and configured to process the fluorescent image of the operative field as it passes through the image transmission system, so as to provide a false-color fluorescent image of the operative field in which a contrast between ligament and bone structures present in the operative field is enhanced relative to the unprocessed fluorescent image; and
an image viewing system, operably connected to the image transmission system, and including a display configured to enable viewing of the false-color fluorescent image of the operative field,
wherein said at least one ligament excitation wavelength includes a wavelength of 395 nm or lower, more preferably 394 nm or lower.

2. The arthroscopic instrument assembly according to claim 1, wherein the tubular housing of the arthroscope at least partially accommodates the illumination system, such that light produced by the light source is emitted from the distal end of the tubular housing.

3. The arthroscopic instrument assembly according to claim 1, wherein said at least one ligament excitation wavelength includes a wavelength in the range 260-300 nm.

4. The arthroscopic instrument assembly according to claim 1, wherein said at least one ligament excitation wavelength includes a wavelength in the range 380-395 nm, more preferably in the range 380-394 nm.

5. The arthroscopic instrument assembly according to claim 1, wherein the image transmission system includes a camera mounted at the distal operative field end of the arthroscope, said camera having at least one image sensor that is operably connected to the image viewing system.

6. The arthroscopic instrument assembly according to claim 5, wherein said at least one image sensor includes an RGB image sensor.

7. The arthroscopic instrument assembly according to claim 5, wherein the image processing system includes at least one optical bandpass filter that is associated with the at least one image sensor and that, seen along an optical path from the operative field to the image sensor, is disposed upstream thereof, said optical bandpass filter being configured to filter at least one emission wavelength from the fluorescent image at which ligament and bone structures present in the operative field have different emission intensities under illumination of light from the light source of the illumination system.

8. The arthroscopic instrument assembly according to claim 7, wherein said at least one emission wavelength includes a wavelength in the range of 400-450 nm.

9. The arthroscopic instrument assembly according to claim 7, wherein said camera has two image sensors, each associated with a respective optical bandpass filter.

10. The arthroscopic instrument assembly according to claim 9, wherein the optical bandpass filter associated with a first of said two image sensors is configured to filter at least one emission wavelength included in the range of 500±20 nm, and
wherein the optical bandpass filter associated with a second of said two image sensors is configured to filter at least one emission wavelength included in the range of 600±20 nm.

11. The arthroscopic instrument assembly according to claim 6, wherein the image processing system is configured to spectrally unmix data received from the at least one image sensor, so as to provide for the false-color fluorescent image.

12. A method of localizing ligament structures within an operative field inside a joint, the method comprising:
illuminating the operative field with light having at least one ligament excitation wavelength;
acquiring and transmitting a fluorescent image of the operative field to an image viewing system;
processing the acquired fluorescent image of the operative field as it is transmitted to the image viewing system, thereby generating a false-color fluorescent image of the operative field in which a contrast between ligament and bone structures present in the operative field is enhanced relative to the unprocessed fluorescent image;
viewing the false-color fluorescent image on the image viewing system, and
localizing the ligament structures present within the operative field in the false-color fluorescent image.

13. The method according to claim 12, wherein said at least one ligament excitation wavelength includes a wavelength in at least one of the ranges 260-300 nm and 380-395 nm.

14. The method according to claim 12, wherein said processing of the acquired fluorescent image includes:
filtering from the fluorescent image at least one emission wavelength in the range of 400-450 nm so as to produce the false-color fluorescent image.

15. The method according to claim 12, wherein said processing of the acquired fluorescent image includes at least one of:
filtering from the fluorescent image at least two emission wavelengths, a first of which is in the range of 500±20 nm, and a second of which is in the range of 600±20 nm, so as to produce at least two filtered fluorescent images; and
filtering from the fluorescent image at least three emission wavelengths, a first of which is in the range of 450-495 nm, a second of which is in the range of 495-570 nm, and a third of which is in the range of 590-750 nm, so as to produce at least three filtered fluorescent images.

16. The method according to claim 12, wherein said processing further includes spectrally unmixing said at least two or three filtered fluorescent images, so as to obtain the false-color fluorescent image.

17. A method of localizing ligament structures within an operative field inside a joint, the method comprising:
illuminating the operative field with light having at least one ligament excitation wavelength;
acquiring and transmitting a fluorescent image of the operative field to an image viewing system;
processing the acquired fluorescent image of the operative field as it is transmitted to the image viewing system, thereby generating a false-color fluorescent image of the operative field in which a contrast between ligament and bone structures present in the operative field is enhanced relative to the unprocessed fluorescent image;
viewing the false-color fluorescent image on the image viewing system, and
localizing the ligament structures present within the operative field in the false-color fluorescent image;
and performing the method using an arthroscopic instrument assembly according to claim 1.
